(19) United States
(12) Patent Application Publication
(10) Pub. No.: US 2008/0207749 A1
(43) Pub. Date: Aug. 28, 2008

(54) Use of Mglur5 (Esp. Afq056) in Gi (Esp.Gerd)

(75) Inventors: Marie-Laure Rouzade-Dominguez, Werentzhouse (FR); Hans-Jurgen Pfannkuche, Weil (DE); Fabrizio Gasparini, Lausen (CH)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: Novartis AG

(21) Appl. No.: 11/995,268

(22) PCT Filed: Jul. 10, 2006
(86) PCT No.: PCT/EP2006/006729

§ 371 Date: Mar. 26, 2008

(30) Foreign Application Priority Data
Jul. 12, 2005 (GB) 0514296.3

(51) Int. Cl. A61K 31/27; A61P 1/04

(52) U.S. Cl. 514/514

(57) ABSTRACT

The invention relates to the use of a compound acting as mGluR5 antagonist in the treatment, preventions and/or delay of progression of disorders of the gastrointestinal tract, disorders of the urinary tract and/or post-operative disorders and to the treatment, prevention and/or delay of progression of pain associated with these indications.

The present invention relates to new pharmaceutical uses of compounds acting as antagonists of metabotropic glutamate type-5 receptors (“mGluR5 antagonists”). The pharmaceutical uses include the treatment, prevention and/or delay of progression of disorders of the gastro-intestinal (GI) tract, disorders of the urinary tract and/or post operative disorders and the treatment, prevention and/or delay of progression of discomfort/pain associated with these indications.

WO 03/047581 discloses mGluR5 antagonists and their use as pharmaceuticals, especially in the treatment of nervous system disorders. WO 05/044265, WO 05/044266, WO 05/044267 disclose mGluR5 antagonists and their use as pharmaceuticals, especially in the treatment or prevention of gastro-esophageal reflux disease (GERD).

The known medication for treatment of disorders of the gastrointestinal and urinary tract has some drawbacks in terms of limited efficacy, tolerability, convenience, and safety.

It has surprisingly found that compounds of formula (I)

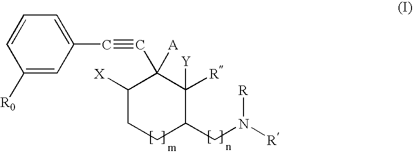

wherein

m is 0 or 1,

n is 0 or 1 and

A is hydroxy

X is hydrogen and

Y is hydrogen, or

A forms a single bond with X or with Y;

R₀ is hydrogen, (C₁₋₄)alkyl, (C₁₋₄)alkoxy, trifluoromethyl, halogen, cyano, nitro, —COOR₁ wherein R₁ is (C₁₋₄)alkyl or —COR₂ wherein R₂ is hydrogen or (C₁₋₄)alkyl, and

R is —COR₃, —COOR₃, —CONR₄R₅ or —SO₂R₆, wherein R₃ is (C₁₋₄)alkyl, (C₃₋₇)cycloalkyl or optionally substituted phenyl, 2-pyridyl or 2-thienyl, R₄ and R₅, independently, are hydrogen or (C₁₋₄)alkyl and R₆ is (C₁₋₄)alkyl, (C₃₋₇)cycloalkyl or optionally substituted phenyl,

R′ is hydrogen or (C₁₋₄)alkyl and

R″ is hydrogen or (C₁₋₄)alkyl, or

R′ and R″ together form a group —CH₂—(CH₂)ₘ—

wherein m is 0, 1 or 2, in which case one of n and m is different from 0, with the proviso that R₀ is different from hydrogen, trifluoromethyl and methoxy when

n is 0, A is hydroxy, X and Y are both hydrogen, R is COOEt and R′ and R″ together form a group —(CH₂)₂—,

in free base or acid addition salt form are highly effective in the treatment, prevention and/or delay of progression of disorders of the gastro-intestinal and urinary tract.

Preferred compounds of formula (I) according to the invention are selected from the following examples:

### EXAMPLE 1

(−)-(3aR,4S,7aR)-4-Hydroxy-4-m-tolylethynyl-octahydro-indole-1-carboxylic acid methyl ester

### EXAMPLE 1a

(−)-(3aR,4S,7aR)-4-Hydroxy-4-m-tolylethynyl-octahydro-indole-1-carboxylic acid ethyl ester

### EXAMPLE 1b

(−)-(3aR,4S,7aR)-Furan-2-yl-(4-hydroxy-4-m-tolylethynyl-octahydro-indol-1-yl)-methanone

### EXAMPLE 1c

(±)-(3aRS,4SR,7aRS)-4-(3-Chlorophenylethynyl)-4-hydroxy-octahydro-indole-1-carboxylic acid ethyl ester

### EXAMPLE 1d

(±)-(3aRS,4SR,7aRS)-4-(3-Fluoro-phenylethynyl)-4-hydroxy-octahydro-indole-1-carboxylic acid ethyl ester

### EXAMPLE 1e

(3aRS,4SR,7aRS)-4-Hydroxy-4-phenylethynyl-octahydro-indole-1-carboxylic acid(S)(tetrahydrofuran-3-yl)ester

### EXAMPLE 1f

(3aRS,4SR,7aRS)-4-Hydroxy-4-phenylethynyl-octahydro-indole-1-carboxylic acid(R)(tetrahydrofuran-3-yl)ester

### EXAMPLE 1g

(3aRS,4SR,7aRS)-4-Hydroxy-4-(3-chlorophenylethynyl)-octahydro-indol-1-carboxylic acid-(S)(tetrahydrofuran-3yl)ester

### EXAMPLE 1h

(±)-(3aRS,4SR,7aRS)-4-Hydroxy-4-m-tolylethynyl-octahydro-indole-1-carboxylic acid ethyl ester

### EXAMPLE 1i

(±)-(3aRS,4SR,7aRS)-4-(4-Fluoro-phenylethynyl)-4-hydroxy-octahydro-indole-1-carboxylic acid ethyl ester

### EXAMPLE 1j

(±)-(3aRS,4SR,7aRS)-4-(3-chlorophenylethynyl)-4-hydroxy-1-methanesulfonyl-octahydro-indole

### EXAMPLE 2

(±)-(3aRS,7aRS)-4-Phenylethynyl-2,3,3a,6,7,7a-hexahydro-indole-1-carboxylic acid ethyl ester and (±)-(RS)-4-phenylethynyl-2,3,5,6,7,7a-hexahydro-indole-1-carboxylic acid ethyl ester

### EXAMPLE 2a

(±)-(3RS,7aRS)-2,2,2-Trifluoro-1-(4-phenylethynyl-2,3,3a,6,7,7a-hexahydro-indol-1-yl)-ethanone

### EXAMPLE 2b

(±)-(RS)-4-m-Tolylethynyl-2,3,5,6,7,7a-hexahydro-indole-1-carboxylic acid ethyl ester

### EXAMPLE 2c

(+)-(3RS,7aRS)-4-m-Tolylethynyl-2,3,3a,6,7,7a-hexahydro-indole-1-carboxylic acid ethyl ester

### EXAMPLE 2d

(±)-(3RS,7aRS)-4-(4-Chloro-phenylethynyl)-2,3,3a,6,7,7a-hexahydro-indole-1-carboxylic acid ethyl ester

### EXAMPLE 2e

(±)-(3RS,7aRS)-4-(2-Fluoro-phenylethynyl)-2,3,3a,6,7,7a-hexahydro-indole-1-carboxylic acid ethyl ester

### EXAMPLE 2f

(±)-(3RS,7aRS)-4-(3-Fluoro-phenylethynyl)-2,3,3a,6,7,7a-hexahydro-indole-1-carboxylic acid ethyl ester

### EXAMPLE 2g

(±)-(RS)-4-(3-Fluoro-phenylethynyl)-2,3,5,6,7,7a-hexahydro-indole-1-carboxylic acid ethyl ester

### EXAMPLE 2h

(±)-(3RS,7aRS)-4-(3-Methoxy-phenylethynyl)-2,3,3a,6,7,7a-hexahydro-indole-1-carboxylic acid ethyl ester

### EXAMPLE 2i

(±)-(RS)-4-(3-Methoxy-phenylethynyl)-2,3,5,6,7,7a-hexahydro-indole-1-carboxylic acid ethyl ester

### EXAMPLE 3

(±)-(3aRS,4RS,7aSR)-4-Hydroxy-4-phenylethynyl-octahydro-isoindole-2-carboxylic acid ethyl ester

### EXAMPLE 3a

(±)-(3aRS,4RS,7aSR)-4-Hydroxy-4-m-tolylethynyl-octahydro-isoindole-2-carboxylic acid ethyl ester

### EXAMPLE 3b

(±)-(3aRS,4RS,7aSR)-4-Hydroxy-4-p-tolylethynyl-octahydro-isoindole-2-carboxylic acid ethyl ester

### EXAMPLE 3c

(±)-(3aRS,4RS,7aSR)-4-(3-Cyano-phenylethynyl)-4-hydroxy-octahydro-isoindole-2-carboxylic acid ethyl ester

### EXAMPLE 3d

(±)-(3aRS,4RS,7aSR)-4-Hydroxy-4-(3-methoxy-phenylethynyl)-octahydro-isoindole-2-carboxylic acid ethyl ester

### EXAMPLE 3e

(±)-(3aRS,4RS,7aSR)-4-(3-Fluoro-phenylethynyl)-4-hydroxy-octahydro-isoindole-2-carboxylic acid ethyl ester

### EXAMPLE 4

(±)-(3aRS,4RS,7aSR)-4-Hydroxy-4-phenylethynyl-octahydro-isoindole-2-carboxylic acid tert-butyl ester

### EXAMPLE 4a

(±)-(3aRS,4RS,7aSR)-4-Hydroxy-4-m-tolylethynyl-octahydro-isoindole-2-carboxylic acid tert-butyl ester

### EXAMPLE 5

(±)-(3aRS,4RS,7aSR)-4-Hydroxy-4-m-tolylethynyl-octahydro-isoindole-2-carboxylic acid methyl ester

### EXAMPLE 5a

(±)-(3aRS,4RS,7aSR)-Furan-2-yl-(4-hydroxy-4-m-tolylethynyl-octahydro-isoindol-2-yl)-methanone

### EXAMPLE 5b

(±)-(3aRS,4RS,7aSR)-Cyclopropyl-(4-hydroxy-4-m-tolylethynyl-octahydro-isoindol-2-yl)-methanone

### EXAMPLE 5c

(±)-(3aRS,4RS,7aSR)-(4-Hydroxy-4-m-tolylethynyl-octahydro-isoindol-2-yl)-pyridin-3-yl-methanone

### EXAMPLE 6

(±)-((1SR,3SR)-3-Hydroxy-3-m-tolylethynyl-cyclohexyl)-methyl-carbamic acid methyl ester and (±)-((1 RS,3SR)-3-hydroxy-3-m-tolylethynyl-cyclohexyl)-methyl-carbamic acid methyl ester

### EXAMPLE 6a

(±)-(1 RS,3SR)-((3-Hydroxy-3-m-tolylethynyl-cyclohexyl)-(4-methoxy-benzyl)-carbamic acid ethyl ester

### EXAMPLE 6b

(±)-(1 RS,3RS)-((3-Hydroxy-3-m-tolylethynyl-cyclohexyl)-(4-methoxy-benzyl)-carbamic acid ethyl ester

### EXAMPLE 6c

(±)-[(1 RS,3SR)-3-Hydroxy-3-(3-methoxy-phenylethynyl)-5,5-dimethyl-cyclohexyl]-methyl-carbamic acid methyl ester.

### EXAMPLE 6d

(±)-(1RS,3SR)-(3-Hydroxy-5,5-dimethyl-3-m-tolylethynyl-cyclohexyl)-methyl-carbamic acid methyl ester

### EXAMPLE 6e

(±)-[(1RS,3SR)-3-(3-Fluoro-phenylethynyl)-3-hydroxy-5,5-dimethyl-cyclohexyl]-methyl-carbamic acid methyl ester

### EXAMPLE 6f

(±)-[(1RS,3RS)-3-(3-Fluoro-phenylethynyl)-3-hydroxy-cyclohexyl]-methyl-carbamic acid methyl ester

### EXAMPLE 6g

(±)-[(1RS,3SR)-3-(3-Fluoro-phenylethynyl)-3-hydroxy-cyclohexyl]-methyl-carbamic acid methyl ester

### EXAMPLE 6h

(±)-[(1RS,3RS)-3-Hydroxy-3-(3-methoxy-phenylethynyl)-cyclohexyl]-methyl-carbamic acid methyl ester

### EXAMPLE 6i

(±)-[(1RS,3SR)-3-Hydroxy-3-(3-methoxy-phenylethynyl)-cyclohexyl]-methyl-carbamic acid methyl ester

### EXAMPLE 6j

(±)-[(1RS,3RS)-3-(3-Chloro-phenylethynyl)-3-hydroxy-cyclohexyl]-methyl-carbamic acid methyl ester

### EXAMPLE 6k

(±)-[(1RS,3SR)-3-(3-Chloro-phenylethynyl)-3-hydroxy-cyclohexyl]-methyl-carbamic acid methyl ester

### EXAMPLE 6l

(±)-(1RS,3RS)-N-(3-hydroxy-3-m-tolylethynyl-cyclohexyl)-acetamide

### EXAMPLE 6m

(±)-(1RS,3SR)-N-(3-hydroxy-3-m-tolylethynyl-cyclohexyl)-acetamide

### EXAMPLE 6n

(±)-(1RS,3RS)-(3-Hydroxy-3-m-tolylethynyl-cyclohexyl)-carbamic acid ethyl ester

### EXAMPLE 6o

(±)-(1RS,3SR)-(3-Hydroxy-3-m-tolylethynyl-cyclohexyl)-carbamic acid ethyl ester

### EXAMPLE 6p

(±)-(1RS,3RS)-[3-(3-Fluoro-phenylethynyl)-3-hydroxy-cyclohexyl]-carbamic acid ethyl ester

### EXAMPLE 6q

(±)-(1RS,3SR)-[3-(3-Fluoro-phenylethynyl)-3-hydroxy-cyclohexyl]-carbamic acid ethyl ester

### EXAMPLE 6r

(±)-(1RS,3RS)-[3-(3-Methoxy-phenylethynyl)-3-hydroxy-cyclohexyl]-carbamic acid ethyl ester

### EXAMPLE 6s

(±)-(1RS,3RS)-N-[3-(3-Fluoro-phenylethynyl)-3-hydroxy-cyclohexyl]-acetamide

### EXAMPLE 6t

(±)-(1RS,3SR)-N-[3-(3-Fluoro-phenylethynyl)-3-hydroxy-cyclohexyl]-acetamide

### EXAMPLE 6u

(±)-(1RS,3SR)-[3-Hydroxy-3-(3-methoxy-phenylethynyl)-cyclohexyl]-carbamic acid ethyl ester

### EXAMPLE 6v

(±)-(1RS,3RS)-N-[3-Hydroxy-3-(3-methoxy-phenylethynyl)-cyclohexyl]-acetamide

### EXAMPLE 6w

(±)-(1RS,3SR)-N-[3-Hydroxy-3-(3-methoxy-phenylethynyl)-cyclohexyl]-acetamide

### EXAMPLE 6x

(±)-(1RS,3RS)-[3-Hydroxy-3-(3-methoxy-phenylethynyl)-cyclohexyl]-carbamic acid tert-butyl ester

### EXAMPLE 6y

(±)-(1RS,3SR)-[3-Hydroxy-3-(3-methoxy-phenylethynyl)-cyclohexyl]-carbamic acid tert-butyl ester

### EXAMPLE 6z

(±)-(1RS,3RS)-(3-Hydroxy-3-m-tolylethynyl-cyclohexyl)-carbamic acid tert-butyl ester

### EXAMPLE 6aa

(±)-(1RS,3SR)-(3-Hydroxy-3-m-tolylethynyl-cyclohexyl)-carbamic acid tert-butyl ester

### EXAMPLE 6ab

(±)-(1RS,3RS)-(3-(3-Fluoro-phenylethynyl)-3-hydroxy-cyclohexyl]-carbamic acid tert-butyl ester

### EXAMPLE 6ac

(±)-(1RS,3SR)-(3-(3-Fluoro-phenylethynyl)-3-hydroxy-cyclohexyl]-carbamic acid tert-butyl ester

### EXAMPLE 6ad

(±)-(1RS,3RS)-[3-(3-Fluoro-phenylethynyl)-3-hydroxy-cyclohexyl]-carbamic acid methyl ester

### EXAMPLE 6ae

(±)-(1RS,3SR)-[3-(3-Fluoro-phenylethynyl)-3-hydroxy-cyclohexyl]-carbamic acid methyl ester

### EXAMPLE 7

(±)-(3-Phenylethynyl-cyclohex-2-enyl)-carbamic acid ethyl ester and (±)-3-phenylethynyl-cyclohex-3-enyl)-carbamic acid ethyl ester

### EXAMPLE 8

(±)-Methyl-(3-phenylethynyl-cyclohex-3-enyl)-carbamic acid ethyl ester

### EXAMPLE 9

(±)-(4aRS,5RS,8aSR)-5-Hydroxy-5-phenylethynyl-octahydro-quinoline-1-carboxylic acid ethyl ester

### EXAMPLE 9a

(±)-[(4aRS,5SR,8aSR)-5-(3-Chloro-phenylethynyl)-5-hydroxy-octahydro-quinolin-1-yl]-furan-2-yl-methanone

### EXAMPLE 9b

(±)-[(4aRS,5RS,8aSR)-5-(3-Chloro-phenylethynyl)-5-hydroxy-octahydro-quinolin-1-yl]-furan-2-yl-methanone

### EXAMPLE 9c

(±)-(4aRS,5RS,8aSR)-5-(3-Chloro-phenylethynyl)-5-hydroxy-octahydro-quinoline-1-carboxylic acid tert-butyl ester

### EXAMPLE 9d

(±)-[(4aRS,5SR,8aSR)-5-(3-Chloro-phenylethynyl)-5-hydroxy-octahydro-quinolin-1-yl]-morpholin-4-yl-methanone

### EXAMPLE 9e

(±)-[(4aRS,5SR,8aSR)-5-(3-chloro-phenylethynyl)-5-hydroxy-octahydro-quinolin-1-yl]-(4-methyl-piperazin-1-yl)-methanone

### EXAMPLE 10

(±)-(4aRS,5RS,8aSR)-5-(3-chloro-phenylethynyl)-5-hydroxy-octahydro-quinoline-1-carboxylic acid ethyl ester and (±)-(4aRS,5SR,8aSR)-5-(3-chloro-phenylethynyl)-5-hydroxy-octahydro-quinoline-1-carboxylic acid ethyl ester

### EXAMPLE 10a

(±)-(4aRS,5SR,8aSR)-5-Hydroxy-5-m-tolylethynyl-octahydro-quinoline-1-carboxylic acid ethyl ester

### EXAMPLE 10b

(±)-(4aRS,5RS,8aSR)-5-Hydroxy-5-m-tolylethynyl-octahydro-quinoline-1-carboxylic acid ethyl ester

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization. The compounds of formula (I) and their manufacture is known, e.g. from WO03/047581.

In accordance with the present invention, it has now surprisingly been found that the compounds of formula (I) are useful in the treatment of disorders of the gastro-intestinal and urinary tract.

In particular compounds of formula (I) are useful in the treatment of conditions associated with visceral hypersensitivity, discomfort/pain and/or altered motor dysfunctions.

Disorders of the GI tract are well known to the expert. These disorders include Gastro-Esophageal Reflux Disease (GERD), Functional Gastro-intestinal Disorders and Post-operative Ileus.

Functional Gastro-intestinal Disorders (FGIDs) are defined as chronic or recurrent conditions associated with abdominal symptoms without organic cause using conventional diagnostic measures. A cardinal symptom present in many FGIDs is visceral pain and/or discomfort. FGIDs include Functional Dyspepsia (FD), functional heartburn (a subset of GERD), Irritable Bowel Syndrome (IBS), Functional Bloating, Functional Diarrhea, Chronic Constipation, Functional Disturbancies of the Biliary Tract as well as other conditions according to Gut 1999; Vol. 45 Suppl. II.

Post-operative Ileus is defined as failure of aboral passage of intestinal contents due to transient impairment of GI motility following abdominal surgery.

Disorders of the Urinary Tract comprise conditions associated with functional disturbancies and/or discomfort/pain of the urinary tract. Examples of disorders of the urinary tract include but are not limited to incontinence, benign prostatic hyperplasia, prostatitis, detrusor hyperreflexia, outlet obstruction, urinary frequency, nocturia, urinary urgency, overactive bladder (OAB), pelvic hypersensitivity, urge incontinence, urethritis, prostatodynia, cystitis, idiopathic bladder hypersensitivity and the like. OAB is a syndrome characterized by urgency, with or without urinary incontinence, and usually with increased voiding frequency and nocturia.

Gastro-Esophageal Reflux Disease (GERD) results from the retrograde flow of gastric contents into the esophagus. It is the most common ailment in the upper gastro-intestinal tract; its cardinal feature and symptom is commonly known as “heartburn”. A major factor considered for GERD is an incompetence of the Lower Esophageal Sphincter that opens transiently and allows passage of material (e.g. meal, acidic fluid or bile), from the stomach into the esophagus. This motor event denominated Transient Lower Esophageal Sphincter Relaxation (TLESR) occurs more often in patients suffering from GERD than in healthy subjects and occurs more often in infants with regurgitation. Current standard therapies in GERD aim at suppressing gastric acid secretion or enhancing gastrointestinal motility to limit the exposure of the esophagus to acidic gastric contents. Frequent exposure of the esophageal mucosa to acid can trigger pain (often perceived as heartburn) and lead to erosions. It can also lead to extra-esophageal disorders such as asthma, cough and laryngitis. To date, there is no treatment available which reduces the occurrence of TLESRs and, thereby, the symptoms associated with GERD or regurgitation in infants.

Functional Dyspepsia (FD) is defined as a condition associated with a heterogeneous pattern of upper abdominal symptoms including discomfort, pain, aching, bloating, belching, fullness, early satiety, nausea and vomiting, burning and indigestion Almost 80% of patients with Functional Dyspepsia have two or more of the above mentioned symptoms of the upper GI tract. The pathophysiological abnormalities observed in FD are as follows: Impaired gastric accommodation upon meal intake, hypersensitivity to gastric distension, delayed gastric emptying, autonomous and/or central nervous system disorder, exaggerated phasic contractile activity, abnormalities of the gastric electrical rhythm, duodenal hypersensitivity to lipids or acid, small intestinal dysmotility. Meals evoke symptoms in more than 75% of FD patients, and symptoms increase with meal ingestion in more than 90% of patients. Therefore, a treatment that prepares the stomach to meal intake has the potential to reduce meal-evoked symptoms. In fact, low fasting volume (not postprandial volume) was found to be an independent predictor for reduced meal size and post-meal symptoms in FD patients (Delgado-Aros et al., Gastroenterology, 2004; 127:1685-1694).

Irritable Bowel Syndrome (IBS) is a chronic or remittent gastrointestinal illness characterized by symptoms that include abdominal pain and/or discomfort, bloating and bowel disturbances, which may be either diarrhea or constipation or a bowel habit that has features of both.

Pain and/or Discomfort is often associated with FGIDs, disorders of the urinary tract and post-operative ileus; it is not only a symptom of GERD. Patients suffering from Irritable Bowel Syndrome (IBS), dyspepsia, diseases of the biliary tract, pancreas, urinary bladder and post-operative conditions report pain and discomfort. Visceral hypersensitivity has been discovered as a key phenomenon in many patients suffering from conditions like IBS, dyspepsia, GERD, functional heartburn and other conditions listed above. To date, there is no medication available which specifically treats visceral hypersensitivity and, thereby, reduces symptoms of pain/discomfort in patients suffering from GERD, functional heartburn, IBS, dyspepsia, diseases of the biliary tract, pancreas, urinary bladder and post-operative conditions. Pain, as used in this specification, includes visceral pain and/or visceral discomfort.

A further aspect of the invention relates to the use of compounds of formula (I) for the treatment of FGIDs (e.g., functional heartburn, FD, IBS).

A further aspect of the invention relates to the use of compounds of formula (I) for the treatment of disorders of the urinary tract.

A further aspect of the invention relates to the use of compounds of formula (I) for the treatment of post-operative ileus.

A further aspect of the invention relates to the use of compounds of formula (I) for the treatment of pain associated with disorders of the gastrointestinal and urinary tract.

A further aspect of the invention relates to the use of compounds of formula (I) for the treatment of altered motor function associated with disorders of the gastro-intestinal and urinary tract.

A further aspect of the invention relates to the use of compounds of formula (I) for the treatment of pain associated with post-operative ileus.

The present invention accordingly provides the use of compounds of formula (I) for treatment of the above-mentioned conditions and disorders.

The present invention accordingly provides the use of compounds of formula (I) for prevention of the above-mentioned conditions and disorders.

The present invention accordingly provides the use of compounds of formula (I) for delay of progression of the above-mentioned conditions and disorders.

A further aspect of the invention relates to the use of compounds of formula (I) for the manufacture of a medicament for the treatment of the above-mentioned conditions and disorders.

A further aspect of the invention relates to a method for the treatment, prevention and /or delay of progression of the above-mentioned conditions and disorders in a subject in need of such treatment, which comprises administering to said subject a therapeutically effective amount of a compound of formula (I) according to claim 1 in free base or pharmaceutically acceptable salt form.

A further aspect of the invention relates to Pharmaceutical composition comprising a compound of formula (I) according to claim 1 in free base or pharmaceutically acceptable salt form for the treatment, prevention and/or delay of progression of the above-mentioned conditions and disorders.

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 0.1 to about 10 mg/kg body weight, e.g. 1 mg/kg. In larger mammals, for example humans, an indicated daily dosage is in the range from about 0.1 to about 1000 mg, preferably from about 1 to about 400 mg, most preferably from about 10 to about 100 mg of a compound of formula (I) conveniently administered, for example, in divided doses up to four times a day.

For use according to the invention, compounds of formula (I) may be administered as single active agent or in combination with other active agents, in any usual manner, e.g. orally, for example in the form of tablets or capsules, or parenterally, for example in the form of injection solutions or suspensions.

Moreover, the present invention provides a pharmaceutical composition comprising a compound of formula (I) in association with at least one pharmaceutical carrier or diluent for use in the treatment of any of the above-indicated diseases. Such compositions may be manufactured in conventional manner. Unit dosage forms may contain, for example, from about 2.5 to about 25 mg of the compound of formula (I).

The present invention also provides the use of a compound of formula (I) for the manufacture of a pharmaceutical composition for the treatment of any of the above-indicated diseases/conditions.

The invention furthermore provides a method for the treatment of any of the above-indicated diseases/conditions, in a subject in need of such treatment, which comprises administering to said subject a therapeutically effective amount of a compound of formula (I).

The effectiveness of the compounds can be shown by a number of well established tests I models, including but not limiting to a GERD model in dogs, a model of fasted gastric tone and gastric accommodation to meal in dogs, visceral hyperalgesia models in rats, a visceral sensation/pain (urinary bladder) model in mice and model of volume-induced contractions of urinary bladder in rats.

GERD Model in Dogs:

Beagle dogs are equipped with a chronic esophagostomy to allow passage of a manometric catheter and a pH probe along the esophagus and the stomach. Following recording of basal pressures of the Lower Esophageal Sphincter and the stomach, one of the novel compound of formula (I) described in WO 03/047581 A1 is administered at doses of 0.03, 0.1, 0.3 and 1 mg/kg i.v. Transient Lower Esophageal Sphincter Relaxations (TLESRs) and acid reflux are induced by infusion of an acidified meal followed by stomach distention using a peristaltic pump infusing air at 40 ml/min, according to a modification of Stakeberg, J. and Lehmann, A. Neurogastroenterol. Mot. (1999) 11: 125-132. Selected compounds of formula (I) reduce dose-dependently the frequency of TLESRs and TLESRs associated with acid reflux. The extent of inhibition obtained for TLESRs as compared to vehicle control was 37% (4.0±0.5, n=6 versus vehicle control 6.4±0.6, n=10, P<0.05) and 62% (2.4±0.2, n=5 versus vehicle control 6.4±0.6, n=10, P<0.01) at doses of 0.3 and 1 mg/kg i.v., respectively. For TLESRs with acid reflux the extent of inhibition as compared to vehicle control was 85% (0.3±0.2, n=6 versus vehicle control 2.1±0.3, n=10, P<0.05) and 70% (0.6±0.4, n=5 versus vehicle control 2.1±0.3, n=10, P>0.05) at doses of 0.3 and 1 mg/kg i.v., respectively

Model of fasted gastric tone and gastric accommodation to meal in dogs: Beagle dogs are equipped with a chronic esophagostomy to allow passage of a balloon (10 cm long, max. volume 950 ml) into the stomach. Approx. 200 ml of air is slowly infused to unfold the balloon, the catheter is retracted to adequately position the balloon in the proximal stomach. After deflation of the balloon, the dog is left undisturbed for 30 min to allow the stomach to return to fasting volume. The balloon is then connected to a barostat device controlling both pressure and volume of the balloon. The minimal distending pressure (MDP) is determined and the balloon pressure is then set at MDP+2 mmHg for 60 min. Afterwards, the pressure is lowered to 0 mmHg while a liquid nutritive meal (20 ml/kg body weight) is infused intragastrically at a speed of 100 ml/min. The balloon is re-inflated at a pressure of MDP+2 mmHg for 60 min while meal-induced gastric accommodation occurs. The compound of formula (I) at a dose of 3 mg/kg or its vehicle is administered orally 10 min after the first balloon distension in the fasting condition. Baseline fasting gastric volume prior to administration was set at 100%. The fasting gastric volume averaged every 10 min (from 0 to 50 min) post vehicle administration amounted to 85%, 81%, 79%, 69% and 68%, respectively. In comparison, a selected compound of formula (I) increased the fasting volume; the averaged volumes (from 0 to 50 min) amounted to 97%, 97%, 159%, 188% and 197%, respectively. One-way repeated measure ANOVA revealed that the gastric volume decreased significantly post vehicle administration (n=5, P<0.01), however, it increased significantly post administration of selected compound of Formula (I) (n=5, P<0.01). Following administration of the meal, gastric volumes were increased significantly to 427% (P<0.05) and 338% (P<0.05) of baseline gastric volume with vehicle and, selected compound of formula (I),.respectively. The results of this study show that the selected compound of formula (I) relaxes the fasted stomach (prior to meal intake) without affecting gastric accommodation as consequence of meal intake. In conclusion, selected compound of formula (I) should be useful for the treatment of dyspeptic symptoms in patients suffering from Functional Dyspepsia, GERD, Functional heartburn and IBS.

Visceral Hyperalgesia in Rats (Model 1):

Trinitrobenzene sulfonic acid (TNBS, 30 mg/kg in ethanol 25%) is instilled in the colon of rats to induce visceral hyperalgesia 7 days prior to experiments applying graded colorectal distensions according to a modified method by Tarrerias, A. et al., Pain (2002) 100: 91-97. Abdominal striated muscle contractions induced by graded colorectal distension (10-60 mmHg, 10 mmHg increments, 3 min duration with 1 min deflation using a barostat) indicate the degree of visceral nociception. Selected compounds of formula (I) at doses of 0.1-10 mg/kg (oral or parenteral administration) reduce the exaggerated abdominal striated muscle contractions, indicative of a visceral antinociceptive activity.

Visceral Hyperalgesia in Rats (Model 2):

In the water avoidance stress model, according to a modification of Schwetz, I. et al., Am. J. Physiol. (2005) 286: G683-G691, selected compounds of formula (I) at doses of 0.1-10 mg/kg (oral or parenteral administration) reduce responses to graded colorectal distension (10-60 mmHg, 10 mmHg increments, 3 min duration with 1 min deflation using a barostat), such as abdominal striated muscle contractions, indicative of a visceral antinociceptive activity.

Visceral Hyperalgesia in Rats (Model 3):

In the model with intracolonic acetic acid (1.5%, 3 days prior to the colorectal distensions), according to a modification of La, J. et al., World J. Gastroenterol. (2003) 9: 2791-2795, selected compounds of formula (I) at doses of 0.1-10 mg/kg (oral or parenteral administration) reduce responses to graded colorectal distension (10-60 mmHg, 10 mmHg increments, 3 min duration with 1 min deflation using a barostat), such as abdominal striated muscle contractions, indicative of a visceral antinociceptive activity.

Visceral Sensation/Pain (Urinary Bladder) in Mice:

An angiocatheter is placed intravesically via the urethra in anesthetized mice and anchored in place. After establishing a reproducible response to urinary bladder distention (UBD; e.g., 60 mm Hg) responses to graded stimuli (10 to 80 mm Hg) are determined according to Ness T J and Elhefni H. J Urol. (2004) 171:1704-8. Electromyographic responses to UBD (electrodes implanted in the superior oblique musculature of the abdomen) are quantified. Selected compounds of formula (I) at doses of 0.1-10 mg/kg (oral or parenteral administration) reduce the EMG (visceromotor) response, indicative of a visceral antinociceptive and/or hypo-sensensitivity activity.

Distension-Induced Contractions of Rat Urinary Bladder:

In anesthetized rats, the urinary bladder was catheterized by use of a PE50 polyethylene tubing filled with physiological saline. Intravesical pressure was measured by a pressure transducer. Cystometry was performed during constant infusion (0.06 ml/min) of saline into the bladder to elicit bladder contractions (Tagaki-Matzumoto et al J. Pharmacol. Sci. (2004) 95 : 458-465). Selected compound of formula (I) at doses of 0.1-10 mg/kg (oral or parenteral administration) increased threshold volumes eliciting bladder contractions indicative of therapeutic potential in conditions with bladder dysfunctions (listed above), e.g., such as overactive bladder, urge incontinence, detrusor hyperreflexia.

These findings with compound of formula (I) are inter alia indicative for the therapeutic use in the treatment of GERD, regurgitation, IBS, dyspepsia, functional bloating, OAB and of conditions associated with visceral discomfort and/or pain.

Post-Operative Ileus Model:

Moreover, it has been found that compound of formula (I) described in WO 03/047581 A1 at doses of 0.01-100 mg/kg, preferably at 0.1-10 mg/kg, e.g., at 1 mg/kg (parenteral adminstration) show activity in the post-operative ileus model according to Huge, A. et al., J. Surg. Res (1998) 74: 112-118, as evidenced by a faster restauration of gastrointestinal motility as compared to vehicle/placebo treatment.

Hence, it follows that compounds of formula (I) are useful in the treatment of post-operative complaints such as visceral pain and/or discomfort, and ileus.

1-8. (canceled)

9. A method for the treatment, prevention and/or delay of progression of disorders of the gastro-intestinal tract, disorders of the urinary tract and/or post operative disorders in a subject in need of such treatment, which comprises administering to said subject a therapeutically effective amount of a compound of formula (I) 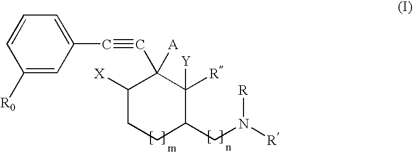
wherein
m is 0 or 1,
n is 0 or 1 and
A is hydroxy
X is hydrogen and
Y is hydrogen, or
A forms a single bond with X or with Y;
R₀ is hydrogen, (C₁₋₄)alkyl, (C₁₋₄)alkoxy, trifluoromethyl, halogen, cyano, nitro, —COOR₁ wherein R₁ is (C₁₋₄)alkyl or —COR₂ wherein R₂ is hydrogen or (C₁₋₄)alkyl, and
R is —COR₃, —COOR₃, —CONR₄R₅ or —SO₂R₆, wherein R₃ is (C₁₋₄)alkyl, (C₃₋₇)cycloalkyl or optionally substituted phenyl, 2-pyridyl or 2-thienyl, R₄ and R₅, independently, are hydrogen or (C₁₋₄)alkyl and R₆ is (C₁₋₄)alkyl, (C₃₋₇)cycloalkyl or optionally substituted phenyl,
R′ is hydrogen or (C₁₋₄)alkyl and
R″ is hydrogen or (C₁₋₄)alkyl, or
R′ and R″ together form a group —CH₂—(CH₂)ₘ—
wherein m is 0, 1 or 2, in which case one of n and m is different from 0,
with the proviso that R₀ is different from hydrogen, trifluoromethyl and methoxy when n is 0,
A is hydroxy, X and Y are both hydrogen, R is COOEt and R′ and R″ together form a group —(CH₂)₂—,
in free base or pharmaceutically acceptable salt form.

10. A method for the treatment, prevention and/or delay of progression of pain associated with disorders of the gastrointestinal tract, the urinary tract and/or post operative disorders in a subject in need of such treatment, which comprises administering to said subject a therapeutically effective amount of a compound of formula (I) according to claim 9 in free base or pharmaceutically acceptable salt form.

11. Pharmaceutical composition comprising a compound of formula (I) according to claim 9 in free base or pharmaceutically acceptable salt form for the treatment, prevention and/or delay of progression of disorders of the gastrointestinal tract, disorders of the urinary tract and/or post operative disorders.

12. Pharmaceutical composition comprising a compound of formula (I) according to claim 9 in free base or pharmaceutically acceptable salt form for the treatment, prevention and/or delay of pain associated with disorders of the gastro-intestinal tract, disorders of the urinary tract and/or post operative disorders.

13. The method according to claim 10 wherein the disorder is selected from the group consisting of gastro-esophageal reflux disease, regurgitation, functional gastro-intestinal disorder, functional dyspepsia, functional heartburn, irritable bowel syndrome, functional bloating, functional diarrhea, chronic constipation, functional disturbancies of the biliary tract, incontinence, benign prostatic hyperplasia, prostatitis, detrusor hyperreflexia, outlet obstruction, urinary frequency, nocturia, urinary urgency, overactive bladder (OAB), pelvic hypersensitivity, urge incontinence, urethritis, prostatodynia, cystitis, and idiopathic bladder hypersensitivity.

14. The method according to claim 9 for the treatment of post-operative ileus or visceral pain and/or discomfort.

15. The method according to claim 9 for the treatment of pain associated with disorders of the gastrointestinal and urinary tract.

16. The method according to claim 9 for the treatment of pain associated with post-operative ileus.

17. The method according to claim 9 for the treatment of conditions associated with visceral hypersensitivity, discomfort/pain and/or altered motor dysfunctions.
